(19) United States
(12) Patent Application Publication
(10) Pub. No.: US 2021/0113774 A1
(43) Pub. Date: Apr. 22, 2021

(54) PARTICLE IMPLANTER CAPABLE OF RAPIDLY SWITCHING IMPLANT NEEDLES

(71) Applicant: FOSHAN BAIKANG ROBOT TECHNOLOGY CO.,LTD., Foshan City (CN)

(72) Inventors: YONGDE ZHANG, FOSHAN CITY (CN); SIHAO ZUO, FOSHAN CITY (CN); RAN WEI, FOSHAN CITY (CN)

(21) Appl. No.: 17/038,967

(22) Filed: Sep. 30, 2020

(30) Foreign Application Priority Data
Oct. 22, 2019 (CN) 201911002867.X

(51) Int. Cl. A61M 5/315; A61M 5/34; A61M 5/31

(52) U.S. Cl. CPC A61M 5/31501; A61M 2205/04; A61M 5/3137; A61M 5/347

(57) ABSTRACT

A particle implanter capable of rapidly switching implant needles is provided, which comprises an inner needle, an outer needle, a stop block A, a connecting rod, a particle library receiving device, a stop block B, a guide rod A, a guide rod B, a slide block, a connecting sleeve, a push block, a handle, a set screw, a positioning screw, a pistol grip, a spring, a positioning shaft, a switch button, a guide sleeve, an implant needle hole and a conical surface. In a switching state, pressing a switching button can gradually reduce a frictional force between a front end of the switching button and the implant needle along an axis the implant needle to zero, thereby rapidly switching the implant needle.

### CROSS REFERENCE TO RELATED APPLICATION

This application claims the priority of Chinese Patent Application No. 201911002867.X entitled “Particle Implanter Capable of rapidly Switching Implant Needles” filed with the Chinese Patent Office on Oct. 22,2019, which is incorporated herein by reference in its entirety.

### TECHNICAL FIELD

The disclosure belongs to the technical field of medical instruments, and particularly relates to a particle implanter capable of rapidly switching implantation needles.

### BACKGROUND ART

Currently, a clinical radioactive particle implantation operation can effectively treat prostate cancer. It is a treatment method in which a doctor uses a particle implanter and imaging apparatus to implant radioactive particles into target regions of a lesion.

However, there are still some problems in a process of using the particle implanter and one of the problems to be urgently solved is how to rapidly switch the implant needles Since a plurality of radioactive particles usually need to be implanted in each particle implantation operation and the target regions are not located at the same position, the implantation needle needs to be replaced many times. Furthermore, due to an influence of manufacturing and machining precision, feeding the implantation needle is often limited, and a phenomenon of “needle collision” may occurs.

### SUMMARY

In view of the above problems, technical problem to be solved by the embodiments is to provide a particle implanter that is able to rapidly switch implant needles.

The disclosure relates to a particle implanter capable of rapidly switching implantation needles, which comprises an inner needle, an outer needle, a stop block A, a connecting rod, a particle library receiving device, a stop block B, a guide rod A, a guide rod B, a slide block, a connecting sleeve, a push block, a handle, a set screw, a positioning screw, a pistol grip, a spring, a positioning shaft, a switch button, a guide sleeve, an implant needle hole, a conical surface. The inner needle and the outer needle are coaxially installed, the outer needle is installed inside the connecting rod and is fixed through a jackscrew, the stop block A is arranged perpendicularly to an axis of the outer needle, the particle library receiving device is arranged perpendicularly to an axis of the connecting rod, the stop block B is connected with the particle library receiving device through the guide rod A and the guide rod B, one end of the guide rod A is installed in a corresponding hole or groove in the stop block A and is fixed through a jackscrew, other end of the guide rod A is installed in a corresponding hole or groove of a slide block and is fixed through a jackscrew, the connecting sleeve is coaxially installed with the guide rod B, one end of the connecting sleeve is installed in the slide block and is fixed through a jackscrew, other end of the connecting sleeve is installed in the push block and is fixed through a jackscrew, the pistol grip and the slide block are positioned through a positioning screw and locked through a set screw, the switch button and the connecting rod are connected through a positioning shaft, and the spring and the connecting rod are connected through welding.

In some embodiments, the guide sleeve is provided with the implant needle pinhole and the conical surface therein.

The beneficial effects of the embodiments are as follows:

(1) Switching implant needles is fast and convenient, switching structure designed in the disclosure is not popularized in the field of medical instruments, the switching part comprises a spring, a positioning shaft, a connecting rod and a switch button, the switch button is connected with the connecting rod through the positioning shaft, the spring is connected with the connecting rod through welding. For convenience of description, one side close to the stop block A is defined as a front end position, and a position close to the pistol grip is defined as a back end position. In a natural state, the switch button is subject to an elastic force of the spring and is constrained within the connecting rod through the positioning shaft; and in a switching state, pressing the switching button can gradually reduce a frictional force between a front end of the switching button and the implant needle along a axis of the implant needle to zero, thereby rapidly switching the implant needle.

(2) The guide sleeve is provided with an implant needle hole and a conical surface, when the implant needle is fed, the implant needle can be conveniently moved to a position at which the implant needle hole is located through the conical surface, so as to prevent the “needle collision” phenomenon in feeding the implant needle, to ensure smooth feeding of the implant needle.

(3) The whole device has a simple structure and is locked by threaded connection, thus is easy to install and disassemble

### BRIEF DESCRIPTION OF THE DRAWINGS

For ease of illustration, the disclosure is described in detail by the following specific embodiments and drawings.

FIG 1 is a schematic view of an overall structure of a particle implanter,

FIG. 2 is a schematic view of a switching portion of a particle implanter,

FIG. 3 is a schematic view of a structure of a guide sleeve.

In the FIGs: 1, an inner needle; 2, an outer needle; 3, a stop block A: 4, a connecting rod; 5 a particle library receiving device; 6, a stop block B; 7, a guide rod A; 8, a guide rod B; 9, a slide block; 10, a connecting sleeve; 11, a push block; 12, a handle; 13, a set screw; 14, a positioning screw; 15, a pistol grip; 16, a spring; 17, a positioning shaft; 18, a switch button; 19, a guide sleeve; 19-1, an implant needle hole; 19-2, a conical surface.

### DETAILED DESCRIPTION OF THE EMBODIMENTS

In order to make objectives, technical solutions and advantages of the disclosure clearer, the disclosure is described below through specific embodiments shown in the drawings. However, it should be understood that these descriptions are only exemplary and not intended to limit the scope of the disclosure. In addition, in the following description, an explanation of well-known structures and technologies are omitted to avoid unnecessarily obscuring a concept of the disclosure.

### Embodiment 1

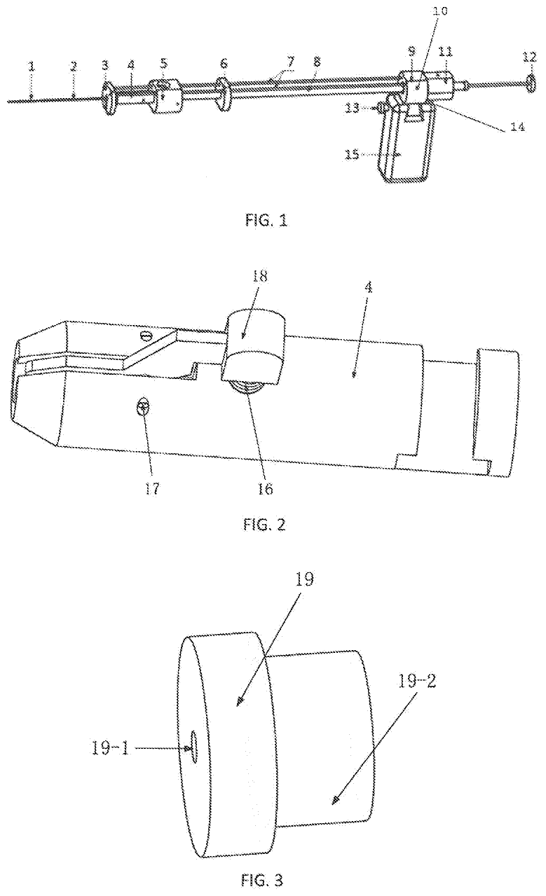

As shown in FIG. 1, FIG. 2 and FIG. 3, the following technical solutions are adopted in specific embodiments of the disclosure. A particle implantcr capable of rapidly switching implant needles, comprises an inner needle 1, an outer needle 2, a stop block A 3, a connecting rod 4, a particle library receiving device 5, a stop block B 6, a guide rod A 7, a guide rod B 8, a slide block 9, a connecting sleeve 10, a push block 11, a handle 12, a set screw 13, a positioning screw 14, a pistol grip 15, a spring 16, a positioning shaft 17, a switch button 18. a guide sleeve 19, an implant needle hole 19-1, a conical surface 19-2. The inner needle 1 and the outer needle 2 are coaxially installed, the outer needle 2 is installed inside the connecting rod 4 and is fixed through a jackscrew, the stop block A 3 is arranged perpendicularly to an axis of the outer needle 2, the particle library receiving device 5 is arranged perpendicularly to an axis of the connecting rod 4, the stop block B 6 is connected with the particle library receiving device 5 through the guide rod A 7 and the guide rod B 8. One end of the guide rod A 7 is installed in a corresponding hole or groove in the stop block A 3 and is fixed through a jackscrcw, other end of the guide rod A 7 is installed in a corresponding hole or groove in the slide block 9 and is fixed through a jackscrcw. The connecting sleeve 10 is coaxially installed with the guide rod B 8, one end of the connecting sleeve 10 is installed in the slide block 9 and is fixed through a jackscrew, other end of the connecting sleeve 10 is installed in the push block 11 and is fixed through a jackscrew, the pistol grip 15 and the slide block 9 are positioned through a positioning screw 14 and locked through a set screw 13. The switch button 18 and the connecting rod 4 are connected through a positioning shaft 17, and the spring 16 and the connecting rod 4 are connected through welding.

Furthermore, the guide sleeve 19 is provided with the implant needle hole 19-1 and a conical surface 19-2.

The above shows and describes basic principle and main features of the disclosure and the advantages of the disclosure. Those skilled in the art should understand that the disclosure is not limited by the embodiments described above, the above-described embodiments and the specification only illustrate the principle of the present disclosure, and various changes and improvements may be made without departing from the spirit and scope of the disclosure, and such changes and improvements are within the scope of the disclosure as claimed. The scope of protection claimed by the disclosure is defined by the appended claims and their equivalents.

What is claimed is:

1. A particle implanter capable of rapidly switching implant needles, comprising: an inner needle, an outer needle, a stop block A, a connecting rod, a particle library receiving device, a stop block B, a guide rod A, a guide rod B, a slide block, a connecting sleeve, a push block, a handle, a set screw, a positioning screw, a pistol grip, a spring, a positioning shaft, a switch button, a guide sleeve, a implant needle hole, a conical surface, wherein the inner needle and the outer needle are coaxially installed, the outer needle is installed inside the connecting rod and is fixed through a first jackscrew, the stop block A is arranged perpendicularly to an axis of the outer needle, the particle library receiving device is arranged perpendicularly to an axis of the connecting rod, the stop block B is connected with the particle library receiving device through the guide rod A and the guide rod B, one end of the guide rod A is installed in a corresponding hole or groove in the stop block A and is fixed through a second jackscrew, other end of the guide rod A is installed in a corresponding hole or groove of the slide block and is fixed through a third jackscrew, the connecting sleeve is coaxially installed with the guide rod B, one end of the connecting sleeve is installed in the slide block and is fixed through a fourth jackscrew, other end of the connecting sleeve is installed in the push block and is fixed through a fifth jackscrew, the pistol grip and the slide block are positioned through the positioning screw and locked through the set screw, the switch button and the connecting rod are connected through a positioning shaft, and the spring and the connecting rod are connected through welding.

2. The particle implanter capable of rapidly switching implant needles according to claim 1, wherein the guide sleeve is provided with the implant needle hole and the conical surface therein.
